(19) United States
(12) Patent Application Publication
(10) Pub. No.: US 2023/0068539 A1
(43) Pub. Date: Mar. 2, 2023

(54) COMPOSITE FILM FRAMEWORK, AND SENSOR, GAS ADSORPTION FILTER, AND GAS REMOVAL DEVICE USING THE COMPOSITE FILM FRAMEWORK

(71) Applicant: Murata Manufacturing Co., Ltd., Nagaokakyo-shi (JP)

(72) Inventor: Hideaki OOE, Nagaokakyo-shi (JP)

(21) Appl. No.: 17/972,632

(22) Filed: Oct. 25, 2022

Related U.S. Application Data
(63) Continuation of application No. PCT/JP2021/022101, filed on Jun. 10, 2021.

(30) Foreign Application Priority Data
Jun. 23, 2020 (JP) 2020-108055

(51) Int. Cl. B01J 20/22; G01N 33/00; B01D 53/02; B01J 20/06; B01J 20/28

(52) U.S. Cl. CPC B01J 20/226; G01N 33/004; B01D 53/02; B01J 20/06; B01J 20/28035; B01D 2253/204; B01D 2253/202; B01D 2253/25; B01D 2257/504

(57) ABSTRACT

A composite film framework that includes a support; a metal oxide layer on the support; and a metal organic framework film on the metal oxide layer 2, wherein a metal atom is shared by a metal oxide in the metal oxide layer and a metal organic framework in the metal organic framework film at an interface between the metal oxide layer and the metal organic framework film.

### CROSS REFERENCE TO RELATED APPLICATIONS

The present application is a continuation of International application No. PCT/JP2021/022101, filed Jun. 10, 2021, which claims priority to Japanese Patent Application No. 2020-108055, filed Jun. 23, 2020, the entire contents of each of which are incorporated herein by reference.

### FIELD OF THE INVENTION

The present invention relates to a composite film framework, and a sensor (particularly, a gas or odor sensor), a gas adsorption filter, and a gas removal device using the composite film framework.

### BACKGROUND OF THE INVENTION

Conventionally, attempts have been made to recover a gas such as carbon dioxide with an adsorbing material. As the adsorbing material, a metal organic framework (MOF), an amine compound, and the like are known (Patent Documents 1 to 3).

For example, Patent Document 1 proposes a method of forming a MOF thin film on a support using a chemical vapor method.

For example, Patent Document 2 proposes a carbon dioxide absorbent in which an amine compound is supported on porous particles in which a hydrophilic fiber and a porous powder are combined with a hydrophilic binder. As the porous powder, MOF is used.

On the other hand, Patent Document 3 proposes a carbon dioxide adsorption substrate in which an amine compound is attached to a porous honeycomb substrate obtained by molding alumina or silica powder.

Patent Document 1: Japanese Translation of PCT International Application Publication No. 2017-519896

Patent Document 2: Japanese Patent Application Laid-Open No. 2018-187574

Patent Document 3: Japanese Translation of PCT International Application Publication No. 2015-508018

### SUMMARY OF THE INVENTION

However, the inventors of the present invention have found that the following new problems arise in the conventional technique.

The technique of Patent Document 1 has a problem that the adhesion of MOF to a support is poor, and the MOF thin film is weak against vibration and a physical load.

In the technique of Patent Document 2, if it is attempted to support MOF on a support such as a honeycomb with a hydrophilic binder, adhesion is not sufficient. Therefore, when an amine compound is supported, a carbon dioxide absorbent is attached to an air conditioner, and the air conditioner is used, the MOF falls off due to vibration or the like, and the carbon dioxide absorption performance is low.

In the technique of Patent Document 3, if a porous honeycomb substrate is used as a support, the carbon dioxide adsorption capability is small because the surface area is not sufficient.

An object of the present invention is to provide a composite film framework in which a metal organic framework (MOF) is supported on a support with sufficient adhesion.

It is also an object of the present invention to provide a sensor (particularly a gas or odor sensor) in which a metal organic framework (MOF) is supported on a support with sufficient adhesion and which is sufficiently excellent in reliability.

It is also an object of the present invention to provide a gas adsorption filter in which a metal organic framework (MOF) is supported on a support with sufficient adhesion and which is sufficiently excellent in gas (for example, carbon dioxide gas) adsorbability.

The present invention relates to a composite film framework including: a support; a metal oxide layer on the support; and a metal organic framework film on the metal oxide layer, wherein a metal atom is shared by a metal oxide in the metal oxide layer and a metal organic framework in the metal organic framework film at an interface between the metal oxide layer and the metal organic framework film.

The present invention also relates to a composite film framework including: a support; a metal oxide layer on the support; and a metal organic framework film on the metal oxide layer, wherein the metal oxide layer has, on at least a surface layer portion thereof, a degenerated layer having a metal oxide of the metal oxide layer that was degenerated by a metal organic framework of the metal organic framework film.

The present invention also relates to a sensor (particularly a gas or odor sensor) including the above-described composite film framework.

The present invention also relates to a gas adsorption filter including the above-described composite film framework.

In the composite film framework of the present invention, a metal organic framework (MOF) is supported on a support with sufficient adhesion. Therefore, each of the sensor and the gas adsorption filter having the composite film framework of the present invention is sufficiently excellent in reliability and gas (for example, carbon dioxide gas) adsorbability.

### BRIEF EXPLANATION OF THE DRAWINGS

FIG. 1A is a schematic sectional view of an example of the composite film framework according to the first embodiment of the present invention.

FIG. 1B is a schematic sectional view of another example of the composite film framework according to the first embodiment of the present invention.

FIG. 2A is a schematic view of a metal organic framework schematically showing a crystal structure of a metal organic framework in the composite film framework according to the first embodiment of the present invention.

FIG. 2B is a schematic view of a metal organic framework schematically showing a crystal structure of a metal organic framework using 2-methylimidazole as an organic molecule in the composite film framework according to the first embodiment of the present invention.

FIG. 2C is a schematic view of a metal organic framework schematically showing a crystal structure of a metal organic framework using a cyan-based organic molecule as an organic molecule in the composite film framework according to the first embodiment of the present invention.

FIG. 3A is a schematic plan view of an example of the gas sensor according to the second embodiment of the present invention.

FIG. 3B is a schematic sectional view of an example of the gas sensor according to the second embodiment of the present invention.

FIG. 3C is a schematic process view illustrating a method of producing the gas sensor according to the second embodiment of the present invention.

FIG. 4A is a schematic plan view of an example of the multi-gas sensor according to the second embodiment of the present invention.

FIG. 4B is a schematic sectional view of an example of the multi-gas sensor according to the second embodiment of the present invention.

FIG. 5 is a schematic view of an example of the gas adsorption filter according to the third embodiment of the present invention.

FIG. 6 is a schematic view of an example of the gas removal device according to the fourth embodiment of the present invention.

FIG. 7 is a graph showing gas adsorbability of a composite film framework produced in examples.

FIG. 8 is a view showing an X-ray diffraction (XRD) spectrum of a composite film framework produced in examples.

### DETAILED DESCRIPTION OF THE INVENTION

### First Embodiment

The first embodiment of the present invention provides a composite film framework. The composite film framework of the present invention includes a support, a metal oxide layer, and a metal organic framework thin film. In the present invention, the metal oxide layer functions as an adhesion layer or an adhesive layer, and the metal organic framework thin film is formed or supported on the support via the metal oxide layer, so that the adhesion of the metal organic framework thin film to the support is sufficiently improved.

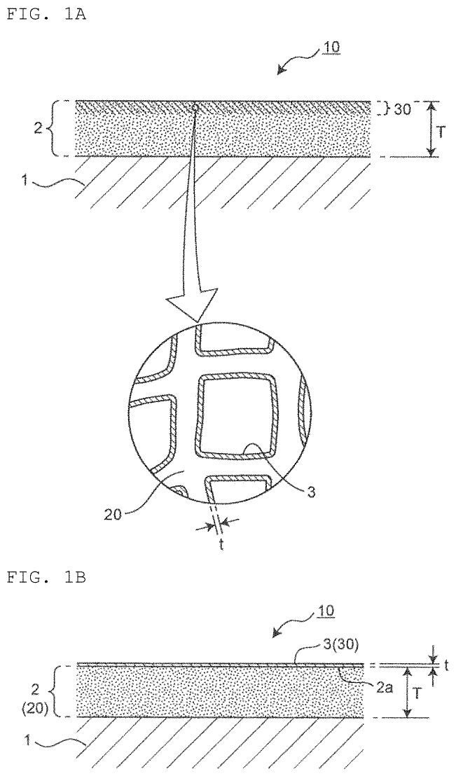

As shown in FIGS. 1A and 1B, for example, a composite film framework 10 of the present invention includes a support 1, a metal oxide layer 2 formed on the support 1, and a metal organic framework thin film 3 formed on the metal oxide layer 2. The “metal organic framework thin film 3 formed on the metal oxide layer 2” means “the metal organic framework thin film 3 included in the metal oxide layer 2”. The “metal organic framework thin film 3 formed on the metal oxide layer 2” includes the following metal organic framework thin films 3 as described in detail below:

In a case where the metal oxide layer 2 is a porous layer (FIG. 1A), the metal organic framework thin film 3 formed on the surface of the metal oxide 20 inside the layer constituting the porous metal oxide layer 2; and

In the case where the metal oxide layer 2 is a non-porous layer (FIG. 1B), the metal organic framework thin film 3 formed on the surface (particularly, the external surface 2a) of the non-porous metal oxide layer 2.

FIG. 1A is a schematic sectional view of an example of the composite film framework according to the first embodiment of the present invention. FIG. 1B is a schematic sectional view of another example of the composite film framework according to the first embodiment of the present invention. In the present specification, various elements in the drawings are merely shown schematically and exemplarily for the understanding of the present invention, and appearance, dimensional ratios, and the like may be different from actual ones. “Vertical direction”, “horizontal direction”, and “front and back direction” used directly or indirectly in the present specification correspond to directions corresponding to the vertical direction, the horizontal direction, and the front and back direction in the drawings, respectively, unless otherwise specified. The same reference sign or symbol indicates the same member or the same meaning except that the shape is different unless otherwise specified.

(Metal Oxide Layer)

The metal oxide layer 2 has, on at least a surface layer portion thereof, a degenerated layer 30. The degenerated layer 30 is a layer in which the metal oxide 20 constituting the metal oxide layer 2 is degenerated by the metal organic framework constituting the metal organic framework thin film 3. The arrangement and form of the degenerated layer 30 vary depending on the form of the metal oxide layer 2. For example, when the metal oxide layer 2 is a porous layer as described later, the degenerated layer 30 is disposed on at least the surface layer portion inside the metal oxide layer 2, and the metal organic framework thin film 3 is formed on the surface of the metal oxide 20 constituting the porous metal oxide layer 2, as shown in FIG. 1A. In this case, the degenerated layer 30 may be disposed over the entire metal oxide layer 2 as long as it is disposed on at least the surface layer portion of the metal oxide layer 2. For example, when the metal oxide layer 2 is a non-porous layer as described later, the degenerated layer 30 is disposed on the surface (particularly, the external surface 2a) of the metal oxide layer 2, and the metal organic framework thin film 3 is formed on the surface of the non-porous metal oxide layer 2, as shown in FIG. 1B. The non-porous layer means a layer having a non-porous (for example, smooth) surface.

In the degenerated layer 30, the metal organic framework of the metal organic framework thin film 3 is configured using metal atoms constituting the metal oxide 20 of the metal oxide layer 2. This is the reason why it is referred to as “degenerated layer”. That is, the degeneration of the “degenerated layer” refers to chemical degeneration of the metal oxide layer 2 (particularly, the metal oxide 20 constituting the layer), and the degenerated layer is a layer in which the metal organic framework of the metal organic framework thin film 3 is formed using metal atoms of the metal oxide 20 constituting the metal oxide layer 2. Specifically, at the interface (or between) between the metal oxide 20 constituting the metal oxide layer 2 and the metal organic framework constituting the metal organic framework thin film 3, metal atoms shared by the metal oxide and the metal organic framework are present. For example, at the interface, metal atoms constituting both the metal oxide and the metal organic framework are present. Further, for example, in the degenerated layer, the metal organic framework is configured while containing metal atoms constituting the metal oxide. As a result thereof, metal atoms are shared by both the metal oxide and the metal organic framework between the metal oxide and the metal organic framework (e.g., at the interface). In the present invention, since the metal organic framework is formed on the surface of the metal oxide while sharing the metal atoms of the metal oxide as described above, the adhesion of the metal organic framework thin film is sufficiently improved.

The material constituting the metal oxide layer 2 is not particularly limited as long as it is a metal oxide capable of supplying a metal atom capable of constituting the metal organic framework, and examples thereof include one or more metal oxides selected from the group consisting of zinc oxide, copper oxide, nickel oxide, iron oxide, indium oxide, and aluminum oxide. The metal oxide layer 2 is preferably made of zinc oxide from the viewpoint of obtaining a porous structure.

The metal oxide layer 2 may be a porous layer or a non-porous layer having a smooth external surface 2a (see FIG. 1B). The metal oxide layer 2 is preferably a porous layer from the viewpoint of carrying more metal organic frameworks based on an increase in the surface area of the metal oxide layer and higher adhesion of the metal oxide layer to the support. For the metal oxide layer 2, the porous layer means a layer having gaps between metal oxide particles (for example, precipitated particles formed by a plating method) constituting the metal oxide layer, or a film or layer having irregularities on the surface.

The thickness T of the metal oxide layer 2 is not particularly limited, and is, for example, 0.1 μm to 100 μm, and is preferably 1 μm to 10 μm from the viewpoint of carrying more metal organic frameworks based on an increase in the surface area of the metal oxide layer and higher adhesion of the metal oxide layer to the support.

As the thickness T of the metal oxide layer 2, the average value of the thicknesses at 50 arbitrary positions in the SEM photograph is used.

The metal oxide layer 2 can be formed by a method such as, for example, a plating method, a CVD method, a vapor deposition method, a sputtering method, or the like. From the viewpoint of increasing the surface area of the metal oxide layer 2, it is preferable to use a plating method. As a method for forming the metal oxide layer by a plating method, an electrolytic plating method and an electroless plating method can be used. In the case of the electrolytic plating method, a conductive material, for example, a metal mesh filter, an activated carbon kneaded filter, or the like can be used as the support. In addition, in the case of a support having low conductivity, it is also possible to form a metal oxide layer by an electroplating method after forming a conductive film made of copper, nickel, or zinc on the surface of the support by an electroless plating method. When the metal oxide layer 2 is a porous layer, the metal oxide layer 2 is preferably formed by a plating method, particularly an electrolytic plating method, from the viewpoint of carrying more metal organic frameworks based on an increase in the surface area of the metal oxide layer and higher adhesion of the metal oxide layer to the support.

(Metal Organic Framework Thin Film)

The metal organic framework thin film 3 is formed of a metal organic framework (That is, MOF: Metal-Organic Framework), and is usually formed only of a metal organic framework. The fact that the metal organic framework thin film 3 is formed only of a metal organic framework means that substances other than the metal organic framework are intentionally not contained, and for example, unintended substances such as metal atoms and organic molecules constituting the metal organic framework, and impurity substances may be contained.

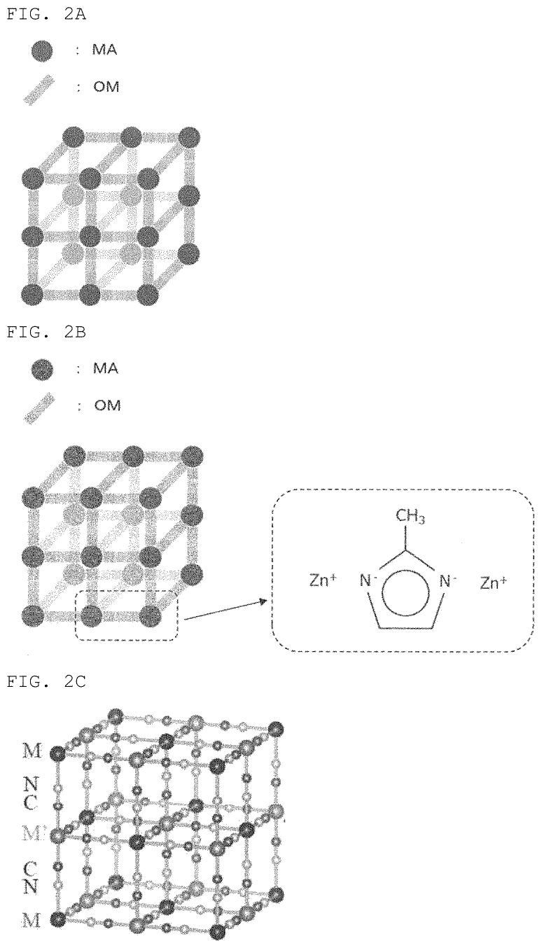

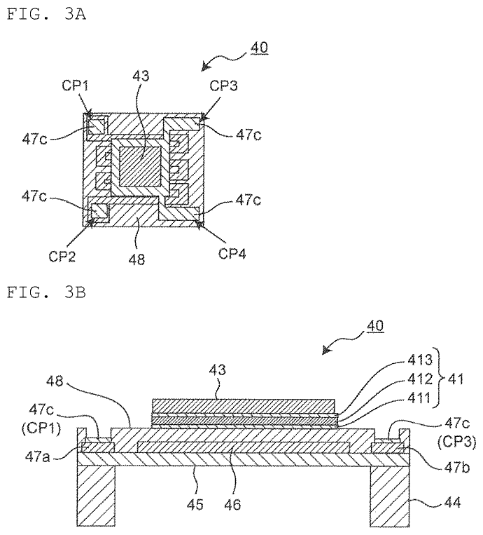

The metal organic framework constituting the metal organic framework thin film 3 is a metal organic framework based on a coordinate bond between organic molecules and metal atoms including metal atoms derived from the metal oxide of the metal oxide layer 2, and configures the metal organic framework thin film 3 as a porous film. The metal organic framework is, for example, a crystalline complex formed by crosslinking a metal atom (particularly a metal atom ion) MA as a ligand with an organic molecule OM, and is a porous body based on a coordinate bond between the organic molecule and the metal atom (particularly a metal atom ion), as shown in FIG. 2A. In the metal organic framework, not all metal atoms constituting the metal organic framework have to be shared by the metal oxide constituting the metal oxide layer 2, but metal atoms of at least the metal organic framework adjacent to the metal oxide (or at least the metal organic framework in the vicinity of the metal oxide) may be shared by the metal oxide. The state in which the metal atom of the metal organic framework adjacent to the metal oxide is shared by the metal oxide can be confirmed by high magnification observation (for example, 1 million times or more) of a transmission electron microscope (TEM). FIG. 2A is a schematic view of a metal organic framework schematically showing a crystal structure of the metal organic framework in the composite film framework according to the first embodiment of the present invention.

Specifically, for example, a metal organic framework containing 2-methylimidazole described later as an organic molecule and a zinc atom as a metal atom may have a crystal structure as shown in FIG. 2B. At this time, one or more of the 18 zinc atoms MA shown in FIG. 2B may be shared by the metal oxide. FIG. 2B is a schematic view of a metal organic framework schematically showing a crystal structure of a metal organic framework using 2-methylimidazole as an organic molecule in the composite film framework according to the first embodiment of the present invention. This structure is merely a schematic view, and the crystal structure is accurately described, for example, in the following documents:

ANH PHAN et al., “Synthesis, Structure, and Carbon Dioxide Capture Properties of Zeolitic Imidazolate Frameworks”(ACCOUNTS OF CHEMICAL RESEARCH 58 67 January 2010 Vol. 43, No. 1)

In addition, for example, a metal organic framework containing a cyan-based compound described later as an organic molecule and containing metal atoms M and M′ may have a crystal structure as shown in FIG. 2C. At this time, any one or more metal atoms among the 26 metal atoms M and M′ excluding the central metal atom M′ shown in FIG. 2C may be shared by the metal oxide. In FIG. 2C, M and M′ may be the same metal atom (e.g., a zinc atom), C is a carbon atom, and N is a nitrogen atom. FIG. 2C is a schematic view of a metal organic framework schematically showing a crystal structure of a metal organic framework using a cyan-based organic molecule as an organic molecule in the composite film framework according to the first embodiment of the present invention.

The organic molecule may be any organic molecule known as an organic molecule capable of constituting a metal organic framework in the field of metal organic frameworks. The organic molecule preferably contains one or more organic molecules selected from the group consisting of azole-based organic molecules, cyan-based organic molecules, and carboxylic acid-based organic molecules from the viewpoint of higher adhesion of the metal organic framework to the support via the metal oxide layer. From the similar point of view and from the viewpoint of further improvement of the detectability (and reliability) of a gas (in particular, a carbon dioxide gas), adsorbability, removability, and structural stability against moisture present in the use environment of each of a sensor, a gas adsorption filter, and a gas removal device using the composite film framework of the present invention, the organic molecules more preferably include one or more organic molecules selected from the group consisting of azole-based organic molecules and cyan-based organic molecules, and further preferably include one or more organic molecules selected from the group consisting of azole-based organic molecules. In the azole-based organic molecule (particularly, the imidazole-based organic molecule) the organic molecule and the metal atom are bonded via a nitrogen atom as shown in FIG. 2B, so the adsorption rate of a gas (particularly, a carbon dioxide gas) is higher.

The azole-based organic molecule constituting the metal organic framework includes an organic molecule selected from the group consisting of imidazole, benzimidazole, triazole, and purine. From the viewpoint of further improvement of the detectability (and reliability) of a gas (in particular, a carbon dioxide gas), adsorbability, and removability of each of a sensor, a gas adsorption filter, and a gas removal device using the composite film framework of the present invention, imidazole, benzimidazole, and purine are preferable, imidazole and benzimidazole are more preferable, and imidazole is further preferable.

The azole-based organic molecule may or may not have a substituent.

Examples of the substituent that may be possessed by the azole-based organic molecule include one or more substituents selected from the group consisting of hydrophobic groups such as an alkyl group, a halogen atom, a nitro group, a phenyl group, a pyridyl group, and a cyano group, and hydrophilic groups such as an amino group and a carboxyl group.

The alkyl group is, for example, an alkyl group having 1 to 5 (particularly 1 to 3) carbon atoms. Specific examples of the alkyl group include a methyl group, an ethyl group, a n-propyl group, an isopropyl group, a n-butyl group, an isobutyl group, a sec-butyl group, a tert-butyl group, and a n-pentyl group.

Examples of the halogen atom include a fluorine atom, a chlorine atom, and a bromine atom.

From the viewpoint of higher adhesion of the metal organic framework to the support via the metal oxide layer and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, the azole-based organic molecule constituting the metal organic framework is preferably selected from the group consisting of azole-based organic molecules having no substituent and azole-based organic molecules having only a hydrophobic group (particularly an alkyl group or a nitro group) if having a substituent, and more preferably selected from the group consisting of azole-based organic molecules having only a hydrophobic group (particularly an alkyl group).

Examples of the azole-based organic molecules constituting the metal organic framework include imidazole-based molecules represented by the following general formula (1), benzimidazole-based molecules represented by the following general formula (2), triazole-based molecules represented by the following general formulas (3) and (4), and purine-based molecules represented by the general formula (5).

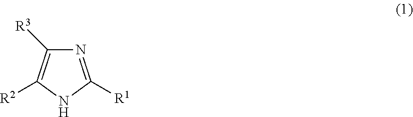

In the formula (1), R¹ to R³ each independently represent a hydrogen atom, a hydrophobic group such as an alkyl group, a halogen atom, a nitro group, a phenyl group, a pyridyl group, or a cyano group, or a hydrophilic group such as an amino group or a carboxyl group, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, a hydrogen atom or the above-described hydrophobic group is preferable, and a hydrogen atom, an alkyl group, a halogen atom, a nitro group, or a cyano group is more preferable. In a more preferred embodiment from the same viewpoint, R¹ is a hydrogen atom, an alkyl group, or a nitro group, and R² and R³ are a hydrogen atom, an alkyl group, a halogen atom, or a nitro group.

Specific examples of the imidazole-based molecule represented by the general formula (1) include the following compounds:

Imidazole, methylimidazole, ethylimidazole, nitroimidazole, aminoimidazole, chloroimidazole, bromoimidazole.

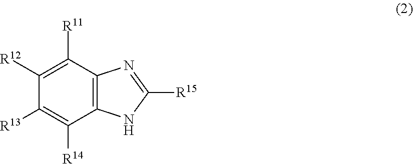

In the formula (2), R¹¹ to R¹⁵ each independently represent a hydrogen atom, a hydrophobic group such as an alkyl group, a halogen atom, a nitro group, a phenyl group, a pyridyl group, or a cyano group, or a hydrophilic group such as an amino group or a carboxyl group, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, a hydrogen atom or the above-described hydrophobic group is preferable, and a hydrogen atom, an alkyl group, a halogen atom, a nitro group, or a cyano group is more preferable. In a more preferred embodiment from the same viewpoint, R¹¹, R¹⁴, and R¹⁵ are a hydrogen atom, and R¹² and R¹³ are each independently a hydrogen atom, an alkyl group, a halogen atom, or a nitro group.

Specific examples of the benzimidazole-based molecule represented by the general formula (2) include the following compounds:

Benzimidazole, chlorobenzimidazole, dichlorobenzimidazole, methylbenzimidazole, bromobenzimidazole, nitrobenzimidazole, aminobenzimidazole, benzimidazole carbonitrile.

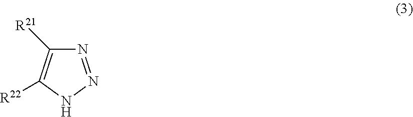

In the formula (3), R²¹ to R²² each independently represent a hydrogen atom, a hydrophobic group such as an alkyl group, a halogen atom, a nitro group, a phenyl group, a pyridyl group, or a cyano group, or a hydrophilic group such as an amino group or a carboxyl group, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, a hydrogen atom or the above-described hydrophobic group is preferable, and a hydrogen atom is more preferable.

Specific examples of the triazole-based molecule represented by the general formula (3) include the following compounds:

1,2,3-triazole.

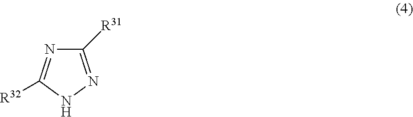

In the formula (4), R³¹ to R³² each independently represent a hydrogen atom, a hydrophobic group such as an alkyl group, a halogen atom, a nitro group, a phenyl group, a pyridyl group, or a cyano group, or a hydrophilic group such as an amino group or a carboxyl group, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, a hydrogen atom or the above-described hydrophobic group is preferable, and a hydrogen atom is more preferable.

Specific examples of the triazole-based molecule represented by the general formula (4) include the following compounds:

1,2,4-triazole.

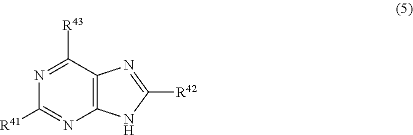

In the formula (5), R⁴¹ to R⁴³ each independently represent a hydrogen atom, a hydrophobic group such as an alkyl group, a halogen atom, a nitro group, a phenyl group, a pyridyl group, or a cyano group, or a hydrophilic group such as an amino group or a carboxyl group, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, a hydrogen atom or the above-described hydrophobic group is preferable, and a hydrogen atom is more preferable.

Specific examples of the purine-based molecule represented by the general formula (5) include the following compounds:

purine.

Examples of the cyan-based organic molecule include potassium ferricyanide, potassium ferrocyanide, and hydrocyanic acid.

As the carboxylic acid-based organic molecule, terephthalic acid, benzenetricarboxylic acid, benzenedicarboxylic acid, or the like can be used.

The metal atom constituting the metal organic framework is a metal atom including a metal atom capable of constituting the metal oxide of the metal oxide layer 2, and is selected from the group consisting of, for example, a zinc atom, a copper atom, a nickel atom, an iron atom, an indium atom, an aluminum atom, a cobalt atom, a praseodymium atom, a cadmium atom, a mercury atom, and a manganese atom, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, the metal atom is preferably selected from the group consisting of a zinc atom, a cobalt atom, and an iron atom, more preferably selected from the group consisting of a zinc atom and a cobalt atom, and still more preferably a zinc atom.

The combination of the organic molecule and the metal atom in the metal organic framework is not particularly limited, but from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, the following combinations are preferred:

Combination (C1)=a combination of an imidazole-based molecule represented by the general formula (1) with one or more metal atoms selected from the group consisting of a zinc atom and an iron atom;

Combination (C2)=a combination of an imidazole-based molecule represented by the general formula (1) with one or more metal atoms selected from the group consisting of a zinc atom and a cobalt atom;

Combination (C3)=a combination of a benzimidazole-based molecule represented by the general formula (2) with one or more metal atoms selected from the group consisting of a zinc atom and a cobalt atom.

The ratio between the organic molecule and the metal atom in the metal organic framework is not particularly limited, but is usually determined by the type of the organic molecule and the type of the metal atom constituting the metal organic framework.

For example, a metal organic framework containing only an imidazole-based molecule (Im) (for example, an imidazole-based molecule represented by the general formula (1)) and one or more divalent metal atoms (M¹) selected from the group consisting of a zinc atom, a cobalt atom, and an iron atom can be represented by the composition formula M¹(Im)₂.

Further, for example, a metal organic framework containing only a benzimidazole-based molecule (bIm) (for example, a benzimidazole-based molecule represented by the general formula (2)) and one or more divalent metal atoms (M¹) selected from the group consisting of a zinc atom, a cobalt atom, and an iron atom can be represented by a composition formula M¹(bIm)₂.

Further, for example, a metal organic framework containing only a triazole-based molecule (Tra) (for example, a triazole-based molecule represented by general formula (3) and/or (4)) and one or more divalent metal atoms (M¹²) selected from the group consisting of a zinc atom, a cobalt atom, and an iron atom can be represented by the composition formula: M¹(Tra)₂.

In addition, for example, a metal organic framework containing only purine-based molecule (Pur) (For example, a triazole-based molecule represented by the general formula (5)) and one or more divalent metal atoms (M¹) selected from the group consisting of a zinc atom, a cobalt atom, and an iron atom can be represented by the composition formula M¹(Pur)₂.

In addition, for example, a metal organic framework containing only an imidazole-based molecule (Im) (for example, an imidazole-based molecule represented by the general formula (1)), a benzimidazole-based molecule (bIm) (for example, a benzimidazole-based molecule represented by the general formula (2)), and one or more divalent metal atoms (M¹) selected from the group consisting of a zinc atom, a cobalt atom, and an iron atom can be represented by a composition formula M¹(Im)ₓ(bIm)_{y} (wherein x+y=2).

The thickness t of the metal organic framework thin film 3 (see FIGS. 1A and 1B) is not particularly limited, and from the viewpoint of higher adhesion of the metal organic framework to the support via the metal oxide layer, and from the viewpoint of further improvement of the detectability (and reliability) of a gas (in particular, a carbon dioxide gas), adsorbability, and removability of each of a sensor, a gas adsorption filter, and a gas removal device using the composite film framework of the present invention, the thickness is preferably 1 nm to 100 nm, and more preferably 10 nm to 100 nm.

As the thickness t of the metal organic framework thin film 3, the average value of thicknesses at 50 arbitrary points in the SEM photograph is used.

The metal organic framework constituting the metal organic framework thin film 3 usually has a pore size of 1 Å to 50 Å. A metal organic framework having an appropriate pore size can be used from the viewpoint of characteristics depending on the application and adhesion to the support. For example, in the case of a sensor using the composite film framework of the present invention, a metal organic framework having a pore size close to the size of the target gas molecule is desirable. In the case of a carbon dioxide sensor, a metal organic framework having a pore size of 2 Å to 5 Å, more preferably 2 Å to 4 Å, which is close to a molecular diameter of carbon dioxide molecule of 3.3 Å, is desirable. In addition, when a polyamine such as polyethyleneimine is supported to form a carbon dioxide adsorption filter, a metal organic framework having a pore size of 5 Å to 20 Å, more preferably 10 Å to 15 Å is preferable in consideration of the unit structure of the polyamine.

The pore size depends on the types of the organic molecule and the metal atom constituting the metal organic framework. Therefore, the pore size can be adjusted by selecting the types of the organic molecule and the metal atom.

In the present specification, the pore size is defined as “When each atom in the crystal is a rigid sphere having a van der Waals radius, the diameter of the largest sphere that can be included”, and is a pore size in a state where no molecule is contained in the pores. Therefore, the pore size can be calculated from the crystal structure. Such a pore size is described as dₚ (Å) in Table 1 of the following document, and the value described in this document can be used.

ANH PHAN et al., “Synthesis, Structure, and Carbon Dioxide Capture Properties of Zeolitic Imidazolate Frameworks” (ACCOUNTS OF CHEMICAL RESEARCH 58 67 January 2010 Vol. 43, No. 1)

The metal organic framework constituting the metal organic framework thin film 3 may be, for example, a metal organic framework shown below.

ZIF-1 (composition formula: Zn(Im)₂);

ZIF-4 (composition formula: Zn(Im)₂);

ZIF-7 (composition formula: Zn(bIm)₂);

ZIF-8 (composition formula: Zn(mIm)₂);

ZIF-9 (composition formula: Co(bIm)₂); ZIF-14 (composition formula: Zn(eIm)₂);

ZIF-81 (composition formula: Zn(cbIm) (nIm));

ZIF-75 (composition formula: Co(mbIm) (nIm));

ZIF-77 (composition formula: Zn(nIm)₂);

ZIF-81 (composition formula: Zn(brbIm) (nIm)).

Here, abbreviations in the composition formula represent the following compounds:

Im: imidazole, bIm: benzimidazole, mIm: methylimidazole, eIm: ethylimidazole, nIm: nitroimidazole, cbIm: chlorobenzimidazole, brbIm: bromobenzimidazole.

The method for forming the metal organic framework thin film 3 is not particularly limited, and specifically, the metal organic framework thin film 3 may be a thin film of (m1) or (m2) formed by the following method.

(m1) A thin film produced by immersing the metal oxide layer in a solution containing organic molecules constituting a target metal organic framework, or

(m2) A thin film produced by immersing the metal oxide layer in a solution containing a target metal organic framework, drying the metal oxide layer, and then mixing an interface between the metal oxide and the metal organic framework by heat treatment.

In the forming method (m1), the organic solvent constituting the solution is not particularly limited as long as it is a solvent capable of dissolving a predetermined organic molecule, and examples thereof include organic solvents such as N,N-diethylformamide, N,N-dimethylformamide and methanol, water, and the like. The formation of the thin film (for example, immersion) may be performed at room temperature or may be performed under heating. Heat and pressure may be applied to thicken the metal organic frameworks. Examples of the method for applying heat and pressure include a method in which the metal oxide layer and the organic molecular solution are heated while being brought into contact with each other in a stainless steel jacket to pressurize the metal oxide layer and the organic molecular solution. The organic molecular concentration of the solution is not particularly limited as long as the metal organic framework can be formed, and is, for example, 10 g/L or more, preferably 20 g/L to 100 g/L, and more preferably 30 g/L to 70 g/L. The heating temperature is not particularly limited as long as the metal organic framework can be formed, and may be, for example, 60° C. or higher and 200° C. or lower, and particularly 130° C. or higher and 150° C. or lower. The heating time is also not particularly limited as long as the metal organic framework can be formed, and may be, for example, 1 hour to 100 hours, and particularly 5 hours to 24 hours. The pressure is not particularly limited as long as the metal organic framework can be formed, and is, for example, a pressure achieved when the above-described organic solvent is heated at the above-described heating temperature for the above-described heating time in a sealed stainless steel jacket, and may be, for example, 1 atm to 2 atm, and particularly 1.2 atm to 1.5 atm. The heating method is not particularly limited, and may be electrical heating or heating by ultrasonic waves or microwaves.

In the forming method (m2), a predetermined metal organic framework is used as the metal organic framework, and for example, a commercially available product of the metal organic framework (for example, the one described above) can be used. Further, for example, the metal organic framework may be deposited on the metal oxide layer by immersing the metal oxide layer in a solution containing a metal ion component and an organic molecular component of the metal organic framework. Here, for example, when ZIF-8 (Zn(mIm)₂) is used as the metal organic framework, zinc nitrate can be used for a metal ion solution, and a 2-methylimidazole solution can be used as an organic molecule solution. As the solvent constituting the solution, the same solvent as the solvent in the forming method (m1) can be used. The mixing is to replace metal atoms in the metal organic framework with metal atoms derived from the metal oxide by heating, and can be achieved, for example, by heating with ultrasonic waves or microwaves.

After the metal organic framework thin film is formed, it is preferable to remove the residual solvent and the adsorption gas by heating. The heating is preferably performed in vacuum (or under a reduced pressure atmosphere).

(Support)

The support 1 is not particularly limited, and may be formed of any substance capable of forming a metal oxide layer. The support is usually formed of one or more materials selected from the group consisting of inorganic materials and organic/polymer materials.

The inorganic material is not particularly limited, and examples thereof include silicon, glass, ceramics, and the like.

The organic/polymer material is not particularly limited, and examples thereof include polyolefins such as polyethylene, polypropylene, and an ethylene-propylene copolymer; polyvinyl chloride; polyvinylidene chloride; polyvinyl acetate; ethylene-vinyl acetate copolymer; polyvinyl alcohol; polyvinyl acetal; fluorine atom-containing polymers such as polyvinylidene fluoride and polytetrafluoroethylene; polyesters such as polyethylene terephthalate, polybutylene terephthalate, polyethylene naphthalate, and polylactic acid; polystyrene; polyacrylonitrile; styrene-acrylonitrile copolymer; ABS resin; polyphenylene ether (PPE); polyimide; amide group-containing polymers such as polyamide or polyamideimide; acrylic polymers such as polyacrylic acid, polymethyl acrylate, polymethacrylic acid, and methyl methacrylate; polycarbonate; polyarylate; polyphenylene sulfide; carbon materials such as carbon, carbon nanotube, and graphene; cellulose nanofibers, and the like.

The support 1 may be made of a metal material or a semiconductor material.

The support 1 may also be made of paper, nonwoven fabric, porous ceramic, resin film, or the like.

The support 1 may further be a crystal oscillator or an oscillator using piezoelectric ceramic, or may be an electrode.

The shape of the support 1 is not particularly limited, and may be, for example, a fibrous shape, a cloth shape, a plate shape, a film shape, or a porous shape (particularly, a honeycomb structure shape).

As the support 1, paper, a nonwoven fabric, porous ceramic, resin film, carbon nanotube, graphene, cellulose nanofiber, or the like can also be used.

When the support 1 has a fibrous shape, the support 1 may be processed into a cloth shape, a plate shape, a film shape, or a honeycomb structure after (1) forming a metal oxide layer on the fibrous support and (2) forming a metal organic framework thin film on the metal oxide layer.

### Second Embodiment

The second embodiment of the present invention provides a sensor using the composite film framework according to the first embodiment. The sensor of the present invention may be a sensor for detecting a gas (in particular a carbon dioxide gas) or odor. In the sensor of the present invention, the adhesion of the metal organic framework thin film to the support is sufficiently improved, similarly to the first embodiment. Therefore, desorption/falling of the metal organic framework thin film is reduced, and a sensor with high adsorbability is obtained, and as a result, a sensor with high reliability (For example, a gas sensor and an odor sensor) can be realized.

In the sensor of the present invention, the metal organic framework thin film can adsorb a large amount of gas due to its porous shape, and the adsorption amount changes depending on the concentration of the surrounding gas. Therefore, the metal organic framework thin film can function as a sensitive film of the gas sensor.

Specifically, since the weight and electrical characteristics of the metal organic framework change due to gas adsorption, the gas adsorption amount can be converted into an electrical signal, that is, a gas sensor can be obtained.

Preferred embodiments of the sensor of the present invention are as follows.

For example, as the support 1 in the first embodiment, it is preferable to use a device whose frequency changes depending on the weight, such as a crystal oscillator or an oscillator using piezoelectric ceramic. A weight change-type gas sensor can be manufactured by forming the metal oxide layer 2 and the metal organic framework thin film 3 on the support 1.

Further, for example, a weight change-type gas sensor can be manufactured by forming a zinc oxide layer (metal oxide layer 2) and a metal organic framework thin film 3 such as the ZIF-8 on a crystal oscillator (support 1) by the method in the first embodiment.

In the present embodiment, the constituent material of the metal oxide layer 2 is not limited to zinc oxide, and may be selected from metal oxides similar to the metal oxides described as the constituent material of the metal oxide layer 2 in the first embodiment.

The constituent material of the metal organic framework thin film 3 can be determined by the gas of interest and the required sensitivity and selectivity. For example, imidazole-based MOFs such as ZIF-1, ZIF-4, ZIF-7, and ZIF-8 can be used as the metal organic framework constituting the metal organic framework thin film 3.

In order to reduce the influence by humidity to improve the accuracy of the sensor, and to obtain appropriate sufficient response speed and recovery speed, a heater (in particular, a heater for heating) may be incorporated to heat the composite film framework.

By arranging a plurality of kinds of metal organic framework materials in an array on different oscillators, it is possible to manufacture a multi-gas sensor capable of simultaneously detecting a plurality of kinds of gases. Such a multi-gas sensor can be an odor sensor.

By forming a piezoelectric thin film and an electrode on a silicon substrate, then forming a heater wiring and a metal organic framework thin film on the metal oxide, and then etching the silicon substrate, a MEMS type gas sensor and order sensor with reduced power consumption can be formed.

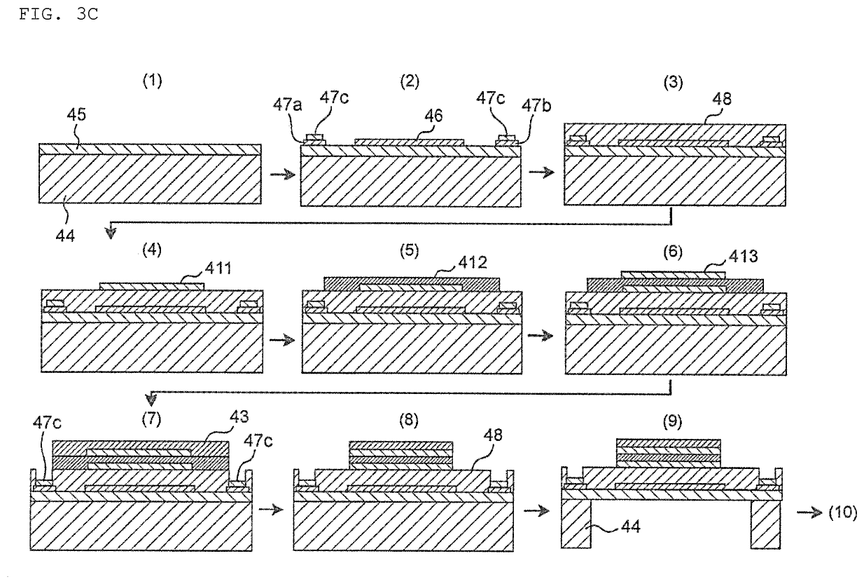

An example of the gas sensor of the present invention is a MEMS type gas sensor illustrated in FIGS. 3A and 3B. FIGS. 3A and 3B are a schematic plan view and a schematic sectional view, respectively, of an example of the gas sensor according to the second embodiment of the present invention.

The gas sensor 40 shown in FIGS. 3A and 3B includes a metal oxide layer (not shown) formed on the piezoelectric oscillator 41 and a metal organic framework thin film 43 carried on the metal oxide layer. In FIG. 3B, the metal oxide layer is omitted. The piezoelectric oscillator 41 corresponds to the support 1 in the first embodiment, and includes a lower electrode 411, a piezoelectric thin film 412, and an upper electrode 413. The metal organic framework thin film 43 corresponds to the metal organic framework thin film 3 in the first embodiment.

The gas sensor 40 usually further includes a silicon substrate 44, a support film 45 formed on the silicon substrate 44, a heater wiring 46 formed on the support film 45, a heater electrode 47a and an oscillator electrode 47b, a wire bonding contact pad 47c formed on the heater electrode 47a and the oscillator electrode 47b, and an insulating layer 48 for insulating the heater wiring 46 and the piezoelectric oscillator 41.

In the gas sensor 40, CP1 is a connection terminal (positive) to the heater, CP2 is a connection terminal (negative) to the heater, CP3 is a connection terminal to the upper electrode of the oscillator, and CP4 is a connection terminal to the lower electrode of the oscillator. The wire bonding contact pad 47c functions as such a connection terminal.

The gas sensor 40 can be produced by, for example, the following method.

Specifically, first, a support film 45 is formed on the silicon substrate 44 (step (1)) as shown in FIG. 3(C). Next, a heater wiring 46, a heater electrode 47a, and an oscillator electrode 47b are formed on the support film 45, and a wire bonding contact pad 47c is formed on the heater electrode 47a and the oscillator electrode 47b (step (2)). Further, an insulating layer 48 is formed, and the heater wiring 46 is insulated from the piezoelectric oscillator 41 to be described later (step (3)). A lower electrode 411 is formed on the insulating layer 48 (step (4)), a piezoelectric thin film 412 is formed on the lower electrode 411 (step (5)), and an upper electrode 413 is formed on the piezoelectric thin film 412 (step (6)). Then, after a metal oxide layer (not illustrated) is formed on the upper electrode 413, a metal organic framework thin film 43 is formed on the metal oxide layer (not illustrated), and a part of the insulating layer 48 is etched to expose the wire bonding contact pad 47c (step (7)). Thereafter, a part of the member on the insulating layer 48 (metal organic framework thin film 43, metal oxide layer (not shown), upper electrode 413, piezoelectric thin film 412, and lower electrode 411) is etched (step (8)), and a part of the silicon substrate 43 is etched (step (9)), so that a sensor 40 can be obtained (step (10)). FIG. 3C is a schematic process view illustrating an example of a method of producing the gas sensor according to the second embodiment of the present invention.

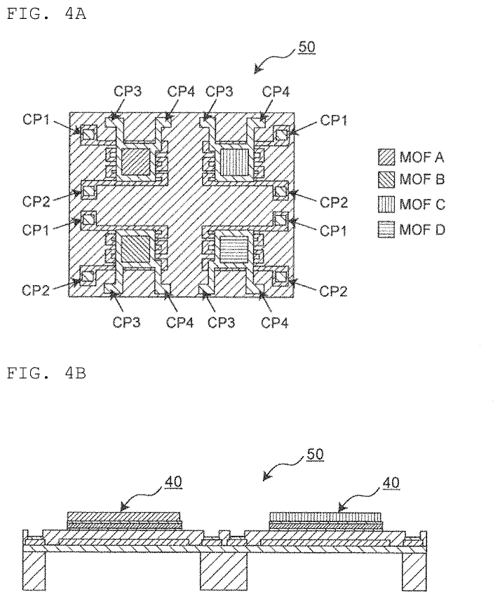

The gas sensor 40 has reduced power consumption. An example of the multi-gas sensor according to the present invention is a MEMS type multi-gas sensor illustrated in FIGS. 4A and 4B. FIGS. 4A and 4B are a schematic plan view and a schematic sectional view, respectively, of an example of the multi-gas sensor according to the second embodiment of the present invention.

The multi-gas sensor 50 illustrated in FIGS. 4A and 4B includes a plurality of (for example, four) gas sensors 40 illustrated in FIGS. 3A and 3B, and the four gas sensors 40 have metal organic framework thin films including different metal organic frameworks.

The method of producing the multi-gas sensor 50 is the same as the method of producing the gas sensor 40 except that a plurality of (for example, four) gas sensors 40 illustrated in FIGS. 3A and 3B are simultaneously produced, and the four gas sensors 40 have metal organic framework thin films including metal organic frameworks different from each other. The metal organic frameworks of the four gas sensors 40 are mutually different metal organic frameworks corresponding to mutually different gases.

The multi-gas sensor 50 has suppressed power consumption. The multi-sensor 50 may function as an odor sensor.

### Third Embodiment

The third embodiment of the present invention provides a gas adsorption filter using the composite film framework according to the first embodiment. The gas adsorption filter of the present invention may be a filter for adsorbing a carbon dioxide gas. In the gas adsorption filter of the present invention, the adhesion of the metal organic framework thin film to the support is sufficiently improved, similarly to the first embodiment. Therefore, a highly reliable gas adsorption filter can be realized.

The gas adsorption filter of the present embodiment has the same structure as the composite film framework according to the first embodiment except that an adsorbing material different from the metal organic framework is attached or supported on the surface of the metal organic framework thin film.

Preferred embodiments of the gas adsorption filter of the present invention are as follows.

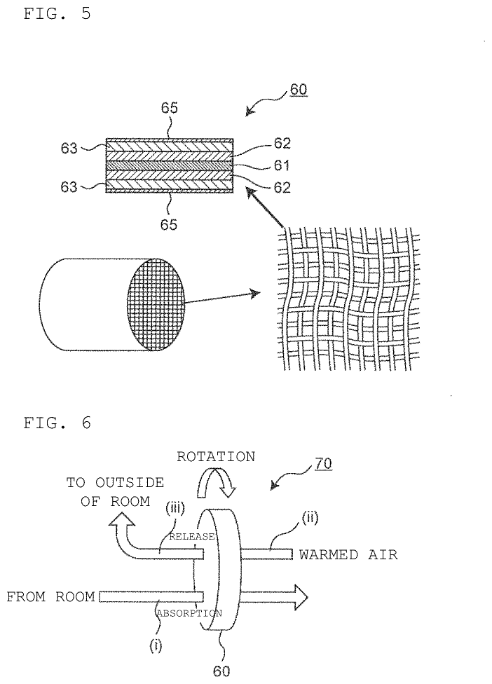

As illustrated in FIG. 5, the gas adsorption filter 60 includes a metal oxide layer 62 formed on a support 61 having a honeycomb structure, a metal organic framework thin film 63 carried on the metal oxide layer 62, and an adsorbing material 65 on the metal organic framework thin film 63. Although the metal organic framework thin film 63 is formed on the external surface of the metal oxide layer 62 in FIG. 5 (see FIG. 1B), the metal organic framework thin film may be disposed on at least the surface layer portion (that is, a degenerated layer (not shown in FIG. 5)) inside the metal oxide layer 62, as shown in FIG. 1A of the first embodiment. For example, the metal organic framework thin film 63 may be formed on the surface of the metal oxide on at least the surface layer portion of the porous metal oxide layer 62. In the present embodiment, the support 61 having a honeycomb structure corresponds to the support 1 in the first embodiment. The metal oxide layer 62 corresponds to the metal oxide layer 2 in the first embodiment. The metal organic framework thin film 63 corresponds to the metal organic framework thin film 3 in the first embodiment. FIG. 5 is a schematic view of an example of the gas adsorption filter according to the third embodiment of the present invention.

By using the support 61 having a honeycomb structure, the surface area of the support itself can be extremely increased. Moreover, as in the first embodiment, the adhesion of the metal organic framework thin film 63 to the support 61 via the metal oxide layer 62 is improved. Therefore, more metal organic frameworks can be attached or carried. Therefore, it is possible to attach or support a larger amount of the adsorbing material 65 while maintaining the adsorption rate of a carbon dioxide gas per unit area. Therefore, the capability of adsorbing a carbon dioxide gas is significantly improved. Also in the present embodiment, since the metal oxide layer 62 acts as an adhesion layer, falling off of the metal organic framework thin film 63 can be sufficiently prevented, and durability is improved.

Specifically, in the present embodiment, the effective surface area in contact with carbon dioxide is extremely increased by a combined effect of an increase in surface area due to the honeycomb structure of the support 61 and an increase in surface area due to surface irregularities and metal organic framework crystals (internal irregularities (that is, pores)) based on the porosity of the metal organic framework thin film 63. As a result, the capability of adsorbing a carbon dioxide gas is significantly improved. Moreover, by making the metal oxide layer porous, the capability of adsorbing a carbon dioxide gas can be further improved.

The adsorbing material 65 is not particularly limited as long as it can adsorb a gas (particularly a carbon dioxide gas), and any adsorbing material used in the field of gas adsorption can be used. As the adsorbing material 65, an amine compound is preferably used from the viewpoint of adsorption of a carbon dioxide gas. The amine compound is not particularly limited as long as it is a substance having an amino group, and an amino group-containing organic compound is usually used. The amino group-containing organic substance is desirably an amino group-containing polymer having a weight average molecular weight of 1,000 or more, preferably 10,000 or more, and more preferably 20,000 or more from the viewpoint of preventing a decrease in the capability of adsorbing a carbon dioxide gas due to volatilization. Specific examples of the amino group-containing polymer include polyethyleneimine, polyamidoamine, and polyvinylamine. The amino group-containing polymer may be linear or branched, and is preferably branched from the viewpoint of further improving the carbon dioxide gas adsorption capability.

The adsorbing material 65 is preferably polyethyleneimine, particularly preferably branched polyethyleneimine, from the viewpoint of further improving the capability of adsorbing a carbon dioxide gas.

The amine value of the amine compound (particularly the amino group-containing polymer) is not particularly limited, and is usually 15 to 25 mmol/gsolid, and from the viewpoint of further improving gas (particularly carbon dioxide gas) adsorbability, the amine value is preferably 17 to 19 mmol/gsolid.

As the amine value, a value measured by a neutralization method calculated from the amount of hydrochloric acid necessary for neutralizing the amine compound is used.

By using an azole-based organic molecule (particularly, an imidazole-based organic molecule) or a cyan-based organic molecule as the organic molecule constituting the metal organic framework thin film, the water resistance of the metal organic framework is improved. Therefore, if an adsorbing material (particularly an amino group-containing polymer) is supported, reliability is higher.

By forming the support 61 into the honeycomb framework, it is possible to improve the carbon dioxide adsorption capability while maintaining the pressure loss.

The gas adsorption filter of the present embodiment can be produced by forming the metal oxide layer 62 (“2” in the first embodiment) and the metal organic framework thin film 63 (“3” in the first embodiment) on the support 61 (“1” in the first embodiment), then removing the residual solvent and the adsorption gas by heating, and attaching or supporting the adsorbing material 65, by the same method as in the first embodiment. The heating is preferably performed in vacuum (or under a reduced pressure atmosphere).

Attaching or supporting of the adsorbing material 65 can be achieved by immersing the composite film framework on which the metal organic framework thin film is formed in an aqueous solution of the adsorbing material (particularly, amine compound) and then drying the framework. As a result, a thin film of the adsorbing material 65 (particularly, the amine compound) may be formed on the metal organic framework thin film.

### Fourth Embodiment

The fourth embodiment of the present invention provides a gas removal device (or a gas removal system) including the gas adsorption filter 60 according to the third embodiment. The gas removal device of the present invention may be a device (or system) for removing a carbon dioxide gas. In the gas removal device of the present invention, the adhesion of the metal organic framework thin film to the support is sufficiently improved, and the capability of adsorbing a carbon dioxide gas can be significantly improved, as in the third embodiment. The present invention makes it possible to realize a small size, energy saving, low cost, and highly reliable gas removal device (particularly, a carbon dioxide gas removal device). The gas removal device of the present invention can also be used for general air conditioning.

As shown in FIG. 6, the gas removal device 70 of the present embodiment can release a carbon dioxide gas in a room to the outside of the room through the following steps. FIG. 6 is a schematic view of an example of the gas removal device according to the fourth embodiment of the present invention.

Step (i):

Air in a room is blown onto the gas adsorption filter 60 to adsorb a carbon dioxide gas.

Step (ii):

By blowing warmed air to the gas adsorption filter 60 or heating the gas adsorption filter 60, the adsorbed carbon dioxide gas is released.

Step (iii):

The released carbon dioxide gas is discharged to the outside of the room.

In the gas removal device 70, the adsorption of carbon dioxide (step (i)) and the release and discharge (steps (ii) and (iii)) may be simultaneously performed by using mutually different positions of the adsorption filter 60, as shown in FIG. 6. At this time, the release position can be changed to the discharge position and the discharge position can be changed to the release position in the adsorption filter 60 by the rotation of the adsorption filter 60. As a result, a carbon dioxide gas can be adsorbed, released, and discharged continuously.

In the gas removal device 70, the adsorption of carbon dioxide (step (i)) and the release and discharge (steps (ii) and (iii)) may be performed in series using the same position of the adsorption filter 60, as an alternative method.

### EXAMPLES

### Experimental Example 1

[Production of composite film framework]

### Example 1

Formation of Metal Oxide Layer

A glass fiber nonwoven support was subjected to ZnO plating. Specifically, the whole surface of the glass support was subjected to electroless Cu plating and electrolytic ZnO plating (thickness of the metal oxide layer: 3 μm) under the following conditions.

Whole surface electroless Cu plating: OPC H-TEC (Okuno Chemical Industries Co., Ltd.), 32° C., 10 minutes; Electrolytic ZnO plating: Zn(NO₃)₂ (Okuno Chemical Industries Co., Ltd.), concentration: 0.1 mol/l, temperature: 60° C., current: 30 mA/dm, time: 2 hours.

Formation of metal organic framework thin film

2.0 g of 2-methylimidazole (Sigma-Aldrich) as an organic molecule was dissolved in 40 ml of N,N-dimethylformamide (Sigma-Aldrich) to obtain an organic molecule solution.

The glass fiber nonwoven fabric support on which the metal oxide layer was formed was placed in a stainless steel jacket together with the above-described organic molecular solution, and heated at 140° C. for 24 hours.

Thereafter, the support was taken out, immersed in methanol, and subjected to ultrasonic cleaning for 10 minutes. The replacement of methanol and the ultrasonic cleaning were repeated 5 times to form a metal organic framework thin film (ZIF-8 (composition formula: Zn(mIm)₂)) on the metal oxide layer (thickness of the metal organic framework thin film: 70 nm).

As a result, a composite film framework as shown in FIG. 1A was obtained.

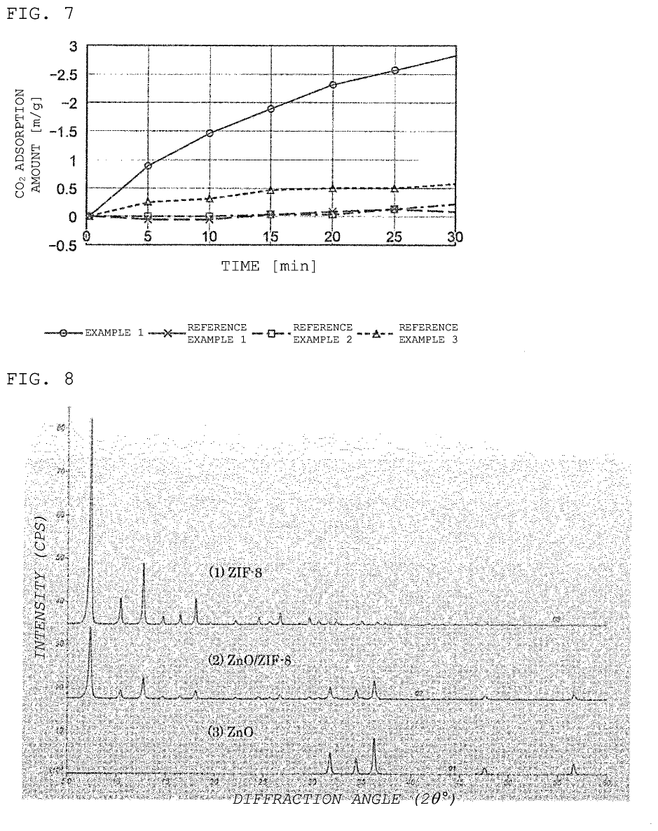

From an X-ray diffraction (XRD) spectrum, it was confirmed that the ZIF-8 thin film was formed on the metal oxide layer. Specifically, as shown in FIG. 8, in the case where the metal organic framework thin film (ZIF-8) was formed on the surface of the zinc oxide layer (ZnO), it was confirmed that the peak of the X-ray diffraction (XRD) spectrum was at the same position as the peak position of the particles of ZIF-8 alone and the peak position of the film of ZnO alone, that is, the composite film framework had both ZIF-8 and ZnO. In FIG. 8, (1) shows an XRD spectrum of particles of a metal organic framework (ZIF-8) alone, (2) shows an XRD spectrum of a film in which a metal organic framework (ZIF-8) is formed on zinc oxide (ZnO), and (3) shows an XRD spectrum of a film of zinc oxide (ZnO) alone.

### Comparative Example 1

4 ml of a ZIF-8 ethanol dispersion liquid having a concentration of 50 g/L was dropped onto the glass fiber nonwoven fabric, and then dried to form a ZIF-8 thin film on the surface of the glass support.

(Evaluation of Adhesion)

The sample obtained in Example 1 or Comparative Example 1 was immersed in pure water, ultrasonic waves were applied at room temperature for 10 minutes, then, the glass support was removed, and 3 cc of the remaining liquid was collected. Using the liquid, the absorbance (absorbance with respect to a water solvent) of light having a wavelength of 550 nm was measured in an optical cell having an optical path length of 1 cm.

No cloudiness was observed in the liquid of Example 1. The absorbance A₅₅₀ of the liquid was less than 0.01, and attenuation of transmitted light was not observed. Therefore, detachment of the MOF thin film was not observed.

In the liquid of Comparative Example 1, cloudiness was observed. The absorbance A₅₅₀ of the liquid was 0.80, and attenuation of transmitted light was observed. From this, it was found that the MOF thin film was detached from the support.

From the above, it was confirmed that the adhesion of the metal organic framework thin film to the support was improved according to the present invention. Here, the absorbance is defined by the following formula based on the light quantity I₀ of the water solvent alone:

$$Absorbance \text{?} = - {log}_{10} ( I / {I}_{0} ) = - \frac{ln ( I / {I}_{0} )}{ln 10} ≃ 0.434 α L$$

$$\text{?} \text{indicates text missing or illegible when filed}$$

I is the light quantity when the solvent is used after the above ultrasonic treatment is performed. (Reference: α is an absorption coefficient, and L is an optical path length (1 cm))

### Experimental Example 2

[Production of Gas Adsorption Filter]

### Example 2

A metal oxide layer (thickness: 3 μm) and a metal organic framework thin film (thickness: 70 nm) were formed on the surface of a support by the same method as in Example 1 except that a ceramic honeycomb structure filter substrate was used as the support.

Polyethyleneimine was dissolved in ethanol to prepare an ethanol solution having a concentration of 60 g/L. 4 mL of an ethanol solution of polyethyleneimine was added dropwise to the support on which the metal oxide layer and the metal organic framework thin film were formed to impregnate the entire filter with polyethyleneimine, and drying was performed to obtain a gas adsorption filter.

### Reference Example 1

A gas adsorption filter was obtained by the same method as in Example 2 except that the metal oxide layer was formed by the following method, and a metal organic framework thin film (MIL-53 (Al)) was formed using terephthalic acid as an organic molecule.

Formation of Metal Oxide Layer

An aluminum oxide thin film having a thickness of 100 nm was formed by an atomic layer volume method. Trimethyl aluminum (Al(CH₃)₃) was used as a precursor gas, water vapor (H₂O+O₂) was used as a reaction gas, and nitrogen (N₂) was used for purging an excess gas. The reaction temperature in the chamber was set to 200° C., the reaction pressure was set to 40 Pa, and the number of cycles was set to 1000.

### Reference Example 2

A gas adsorption filter was obtained by the same method as in Example 2 except that the metal oxide layer was formed by the following method, and a metal organic framework thin film (Cu-BTC) was formed using trimesic acid as an organic molecule.

Formation of Metal Oxide Layer

Whole surface electroless Cu plating: OPC H-TEC (Okuno Chemical Industries Co., Ltd.), 32° C., 10 minutes;

Electrolytic ZnO plating: CuSO₄ (Sigma-Aldrich), concentration: 0.4 mol/l, temperature: 32° C., current: 100 mA/dm, time: 2 hours.

### Reference Example 3

A gas adsorption filter was obtained by the same method as in Example 2 except that a metal organic framework thin film (MOF-177 (Zn)) was formed using 1,3,5-tris(4-carboxyphenyl)benzene as an organic molecule.

(Evaluation of Carbon Dioxide Gas Adsorption Amount)

The gas adsorption filter obtained in each of Example 2 and Reference Examples 1 to 3 was placed in an acrylic case (12 L). By monitoring the concentration of a carbon dioxide gas in the acrylic case, the temporal change in the adsorption amount of the carbon dioxide gas was measured and shown in the graph of FIG. 7.

Measurement of adsorption of CO2

After heating at 60° C. for 60 minutes, the CO2 concentration was monitored at 25° C. and 40% RH.

Measurement of Detachment of CO2

CO2 adsorption was saturated at 25° C. and 40% RH, followed by monitoring the CO2 concentration at 60° C. and 10% RH.

From the measured value of the carbon dioxide gas concentration, the carbon dioxide gas adsorption amount was calculated using the following formula, and plotted with respect to time:

$${Co}_{2} Adsorption amount [ ml / g ] = \frac{Δ C [ ppm ] \times {10}^{- 6} \times V [ L ] \times {10}^{3}}{w [ g ]}$$

In the formula, the abbreviations denote the following meanings.

ΔC: reduction amount of the carbon dioxide concentration from start of measurement (difference between the carbon dioxide concentration and the carbon dioxide concentration at start of measurement)

V: volume of acrylic case

w: difference between the weight of the carbon dioxide adsorption filter and the weight of the filter support (that is, the sum of the weights of the metal oxide, the metal organic framework, and the amine compound)

The composite film framework of the present invention is useful for a sensor (particularly, a gas or odor sensor), a gas adsorption filter, and a gas removal device.

### DESCRIPTION OF REFERENCE SYMBOLS

1: Support

2: Metal oxide layer

3: Metal organic framework thin film (MOF thin film)

10: Composite film framework

20: Metal oxide

30: Degenerated layer

40: Gas sensor

50: Multi-gas sensor

60: Gas adsorption filter

61: Support

62: Metal oxide layer

63: Metal organic framework thin film (MOF thin film)

65: Adsorbing material

70: Gas removal device

1. A composite film framework comprising:
a support;
a metal oxide layer on the support; and
a metal organic framework film on the metal oxide layer,
wherein the metal oxide layer has, on at least a surface layer portion thereof, a degenerated layer having a metal oxide of the metal oxide layer that was degenerated by a metal organic framework of the metal organic framework film.

2. The composite film framework according to claim 1, wherein in the degenerated layer, the metal organic framework film is on a surface of the metal oxide.

3. The composite film framework according to claim 1, wherein in the degenerated layer, the metal organic framework is configured using metal atoms constituting the metal oxide.

4. The composite film framework according to claim 1, wherein the metal oxide layer is a porous layer.

5. The composite film framework according to claim 1, wherein the metal organic framework includes an organic molecule, and the organic molecule is one or more selected from the group consisting of azole-based organic molecules, cyan-based organic molecules, and carboxylic acid-based organic molecules.

6. The composite film framework according to claim 5, wherein the organic molecule comprises an azole-based organic molecule selected from the group consisting of imidazole, benzimidazole, triazole, and purine.

7. A composite film framework comprising:
a support;
a metal oxide layer on the support; and
a metal organic framework film on the metal oxide layer,
wherein a metal atom is shared by a metal oxide in the metal oxide layer and a metal organic framework in the metal organic framework film at an interface between the metal oxide layer and the metal organic framework film.

8. The composite film framework according to claim 7, wherein the metal oxide layer is a porous layer.

9. The composite film framework according to claim 7, wherein the metal oxide layer is a porous plating layer.

10. The composite film framework according to claim 7, wherein the metal oxide layer is a layer containing one or more metal oxides selected from the group consisting of zinc oxide, copper oxide, nickel oxide, iron oxide, indium oxide, and aluminum oxide.

11. The composite film framework according to claim 7, wherein the metal oxide layer has a thickness of 1 μm to 10 μm.

12. The composite film framework according to claim 7, wherein the metal organic framework includes an organic molecule, and the organic molecule is one or more selected from the group consisting of azole-based organic molecules, cyan-based organic molecules, and carboxylic acid-based organic molecules.

13. The composite film framework according to claim 12, wherein the organic molecule comprises an azole-based organic molecule selected from the group consisting of imidazole, benzimidazole, triazole, and purine.

14. The composite film framework according to claim 7, wherein the metal organic framework film has a thickness of 10 nm to 100 nm.

15. A gas or odor sensor comprising the composite film framework according to claim 7.

16. The gas or odor sensor according to claim 15, wherein the support is a crystal oscillator or a piezoelectric ceramic.

17. A gas adsorption filter comprising the composite film framework according to claim 7.

18. The gas adsorption filter according to claim 17, wherein an amine compound is supported on the metal organic framework film, and the gas is a carbon dioxide gas.

19. The gas adsorption filter according to claim 18, wherein the amine compound is an amino group-containing polymer having a weight average molecular weight of 1000 or more.

20. The gas adsorption filter according to claim 17, wherein the organic molecule is an azole-based organic molecule or a cyan-based organic molecule.
